(19) United States
(12) Patent Application Publication
(10) Pub. No.: US 2026/0345917 A1
(43) Pub. Date: Nov. 5, 2026

(54) DRUG INJECTION DEVICE

(71) Applicant: SHL Medical AG, Zug (CH)

(72) Inventor: Nurettin Ali, Deerfield Beach, FL (US)

(21) Appl. No.: 19/119,899

(22) PCT Filed: Sep. 28, 2023
(86) PCT No.: PCT/EP2023/076990

§ 371 Date: Apr. 10, 2025

Related U.S. Application Data
(60) Provisional application No. 63/415,322, filed on Oct. 12, 2022.

(30) Foreign Application Priority Data
Nov. 15, 2022 (EP) 22207423.9

(51) Int. Cl. A61M 5/32; A61M 5/24

(52) U.S. Cl. CPC A61M 5/3202; A61M 5/24; A61M 2005/2418

(57) ABSTRACT

The present disclosure provides a drug injection device, comprising a cap and a housing, said cap being removably attached to a proximal end of the housing. The housing includes a support configured for axially abut a flange of a syringe. The cap comprises at least one flexible rib extending axially, a distally facing surface of said flexible rib abutting a proximally facing surface of the housing.

### TECHNICAL FIELD

The present disclosure generally relates to the technical field of drug injection devices.

### BACKGROUND

Injection devices, such as auto-injectors, are known in the art for dispensing a medicament to the injection site of a patient. Such injection devices typically comprise a housing and a cap, the cap being located at a front side of the device. A syringe is located in the housing and is sometimes covered by a needle shield. The cap and needle shield are removably attached to the housing to shield or expose the needle of the syringe. To dispense the medicament, the cap and needle shield, initially shielding the needle, are first removed from the housing to expose the needle. The needle is then inserted into the housing of the patient at the injection site to dispense the medicament.

Often, such injection devices are required to work with glass pre-filled syringes. Such glass syringes have a cylindrical barrel that holds the medicament to be injected, along with a flange at their base.

The barrel of the syringe is classically housed inside a syringe carrier. A conventional carrier provides shoulders that are adapted to engage a shoulder on the syringe barrel and prevent the syringe from disengaging the carrier. The syringe carrier is normally designed to take up forces applied to the syringe to prevent damage to the frangible glass housing of the syringe.

In order to use one less component, thereby simplifying manufacture of the device, the assembly may not use any syringe carrier. In that case, the housing may comprise one or several support elements holding the syringe by its flange within the housing. In this case, the flange of the syringe axially abuts the support element(s) of the housing. However, assembling the syringe without using any syringe carrier when the syringe is held by its flange within the housing poses a risk of breaking the flange when the injection device is dropped front end first before use. Hence, a technical problem to solve is how to dampen axial shocks against the cap in order to avoid damages to the glass syringe located inside the housing, the syringe having a flange axially abutting the support element(s) of the housing.

### SUMMARY

To help solve that problem, in a first embodiment, the present disclosure provides a drug injection device comprising a cap and a housing, said cap being removably attached to a proximal end of the housing, the proximal end referring to the end which, during use of the drug injection device, is located closest to the drug delivery site, the housing comprising a support configured for axially abut a flange of a syringe, axial referring to a longitudinal axis of the drug injection device, wherein the cap comprises at least one flexible rib extending axially, a distally facing surface of said flexible rib abutting a proximally facing surface of the housing.

In a second embodiment, the present disclosure provides a drug injection device comprising a cap and a housing, said cap being removably attached to a proximal end of the housing, the proximal end referring to the end which, during use of the drug injection device, is located closest to the drug delivery site, the housing comprising a support configured for axially abut a flange of a syringe, axial referring to a longitudinal axis of the drug injection device, wherein the housing comprises at least one flexible rib extending axially, a proximally facing surface of said at least one flexible rib abutting a distally facing surface of the cap.

The technical problem is solved by providing a dampening achieved by an axial coupling of the cap to the housing via a resilient interface area. In the first and second embodiments, the resilient interface area is made of one or several flexible ribs, integral with the cap or with the housing, that is/are configured to deform in case of a front end drop of the drug. Consequently, if a drug injection device according to the invention drops front end first, that is to say the side where the cap is attached, also called proximal side, the energy from the shock is absorbed by the rib, thereby preventing the energy from being transmitted from the cap to the housing and thus to the syringe held by the flange within the housing. It should be noted that the flexible rib(s) may flex radially inward or radially outward.

Preferred embodiments of the drug injection device are indicated in the appended subclaims.

Preferably, there is more than one such flexible rib, said flexible ribs being circumferentially distributed around the longitudinal axis.

Preferably, there are four such flexible ribs equally distant from each other.

In a third embodiment, the present disclosure provides a drug injection device comprising a cap and a housing, said cap being removably attached to a proximal end of the housing, the proximal end referring to the end which, during use of the drug injection device, is located closest to the drug delivery site, the housing comprising a support configured for axially abut a flange of a syringe, axial referring to a longitudinal axis of the drug injection device, wherein the drug injection device comprises a resilient ring located between a distally facing surface of the cap and a proximally facing surface of the housing.

The technical problem is solved by providing a dampening achieved by an axial coupling of the cap to the housing via a resilient interface area. In the third embodiment, the resilient interface area is made of the resilient ring that is configured to compress in case of a front end drop of the drug injection device. Consequently, if a drug injection device according to the invention drops front end first, that is to say the side where the cap is attached, also called proximal side, the energy from the shock is absorbed by the resilient ring, thereby preventing the energy from being transmitted from the cap to the housing and thus to the syringe held by the flange within the housing.

Preferred embodiments of the drug injection device are indicated in the appended subclaims.

Preferably, the resilient ring is made of rubber or thermoplastic elastomer.

In a sub-embodiment, the cap comprises a distal portion shrouding a proximal portion of the housing, and the ring is radially biased against said distal portion of the cap, radially being relative to the longitudinal axis.

In another sub-embodiment, the housing comprises a proximal portion shrouding a distal portion of the cap, and the ring is radially biased against said proximal portion of the housing, radially being relative to the longitudinal axis.

In an embodiment, the drug injection device comprises a syringe located within the housing and having a flange held by the support of the housing, said flange axially abutting the support.

In the present disclosure, when the term “distal direction” is used, this refers to the direction pointing away from the drug delivery site during use of the drug injection device. When the term “distal part/end” is used, this refers to the part/end of the injection device, or the parts/ends of the members thereof, which during use of the drug injection device is/are located furthest away from the drug delivery site. Correspondingly, when the term “proximal direction” is used, this refers to the direction pointing towards the drug delivery site during use of the drug injection device. When the term “proximal part/end” is used, this refers to the part/end of the injection device, or the parts/ends of the members thereof, which during use of the drug injection device is/are located closest to the drug delivery site.

Further, the terms “longitudinal”, “longitudinally”, “axially” and “axial” refer to a direction extending from the proximal end to the distal end and along the device or components thereof, typically in the direction of the longest extension of the device and/or component.

Similarly, the terms “transverse”, “transversal” and “transversally” refer to a direction generally perpendicular to the longitudinal direction.

Generally, all terms used in the claims are to be interpreted according to their ordinary meaning in the technical field, unless explicitly defined otherwise herein. All references to a/an/the element, apparatus, member, component, means, etc. are to be interpreted openly as referring to at least one instance of the element, apparatus, member component, means, etc., unless explicitly stated otherwise.

### BRIEF DESCRIPTION OF THE DRAWINGS

Embodiments of the present disclosure will now be described by way of example only and with reference to the following accompanying drawings.

FIG. 1 shows a cross-sectional view of some parts of a drug injection device according to a first embodiment of the invention.

FIG. 2A shows a perspective view of a cap of the drug injection device of FIG. 1.

FIG. 2B shows an end view of the cap of FIG. 2A.

FIG. 2C shows a cross-sectional view of the cap of FIG. 2C.

FIG. 3 shows a close-up view of a proximal part of the drug injection device of FIG. 1.

FIG. 4 shows a cross-sectional view of a proximal part of a drug injection device according to a second embodiment of the invention.

FIG. 5 shows a simplified schematic view of a drug injection device according to a third embodiment of the invention.

FIG. 6 shows a simplified schematic view of a drug injection device according to a fourth embodiment of the invention.

### DETAILED DESCRIPTION

Embodiments of the present disclosure will now be described by way of example only and with reference to the accompanying drawings. The invention is defined by the appended claims, to which reference should now be made.

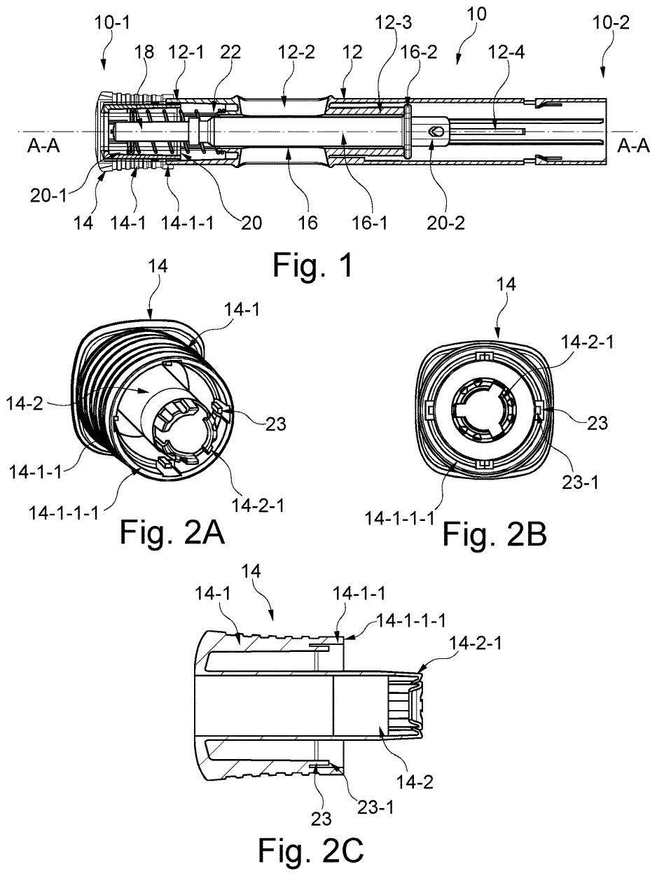

FIG. 1 shows a cross-sectional view of a drug injection device 10 according to a first embodiment of the invention. The drug injection device 10 is in the present example an autoinjector. The drug injection device 10 extends along a longitudinal axis A-A between a proximal side 10-1, where the drug is ejected, and a distal side 10-2.

The drug injection device 10 comprises a housing 12. The housing 12 is hollow and may be generally tubular, although other shapes are possible. The housing 12 extends along the longitudinal axis A-A between the proximal side 10-1 and the distal side 10-2. The housing 12 comprises a proximal portion 12-1, generally annular but which could be of any other shape. The housing 12 also comprises an optional window 12-2 through which a user can see the drug to be injected.

The drug injection device 10 comprises a cap 14, shown in more details in FIGS. 2A to 2C. The cap 14 can be fitted to the housing 12 for removable attachment of the cap 14 to the housing 12. For that purpose, the cap 14 comprises a shell 14-1 having a distal portion 14-1-1, generally annular, configured to shroud the proximal portion 12-1 of the housing 12. It should be noted that the distal portion 14-1-1 of the shell 14-1 of the cap 14-1 and the proximal portion 12-1 of the housing 12 may alternatively be non-circular, as long as they have corresponding shapes. Thus, the cap 14 and the housing 12 may be assembled using a snap-fit or a friction-fit, for instance. The cap 14 and the housing 12 form a sub-assembly for a drug delivery device, according to the invention.

The drug injection device 10 is configured to accommodate a drug container such as a syringe 16. The syringe 16 comprises a barrel 16-1, generally cylindrical, that holds the drug to be injected, a flange 16-2 on the distal side, and a delivery member on the proximal side, e.g., a needle or a nozzle. The barrel 16-1 and the flange 16-2 are generally made of glass. The invention is particularly adapted to a drug injection device 10 having a flange 16-2 made of glass, or a flange made of any other material that can be broken as a result of a shock.

The delivery member may for example be fitted with a flexible delivery a member shield, e.g., a flexible needle shield (FNS) and/or a rigid delivery member shield 18, e.g., a rigid needle shield (RNS) or both. In one example where the delivery member is fitted with both the delivery member shield and the rigid delivery member shield 18, the rigid delivery member shield 18 may be arranged around the flexible delivery member shield.

The cap 14 comprises a delivery member shield remover 14-2 configured to engage with the rigid delivery member shield 18. The delivery member shield remover 14-2 may for example comprise radially inwards extending tabs 14-2-1 configured to engage with the rigid delivery member shield 18. When the cap 14 is removed from the housing 12, it brings with it the flexible delivery member shield and the rigid delivery member shield 18. In the present embodiment, the delivery member shield remover 14-2 is integral with the shell 14-1, however the delivery member shield remover 14-2 may alternatively be a separate piece that can be assembled to the shell 14-1.

The housing 12 comprises a support 12-3, the support 12-3 being an integral part of the housing 12. The support 12-3 is configured to hold the flange 16-2 of the syringe 16, preventing the syringe 16 from moving axially in the proximal direction, i.e. towards the proximal side 10-1 of the drug injection device 10.

The drug injection device 10 comprises a delivery member cover 20. The delivery member cover 20 is slidably arranged in the housing 12 and protrudes from a proximal end opening of the housing 12. The delivery member cover 20 is configured to move axially relative to the housing 12 from an extended position to a retracted position in which the housing 12 receives a larger portion of the delivery member cover 20 than in the extended position. The delivery member cover 20 has a proximal tubular portion 20-1 and legs 20-2 extending distally towards the distal end of the housing 12. The housing 12 comprises guide rails 12-4. The legs 20-2 are configured to cooperate with the guide rails 12-4 when the delivery member cover 20 is moved between the extended position and the retracted position.

In a preferred example, the drug injection device 10 comprises a resilient member 22 configured to bias the delivery member cover 20 towards the extended position. The resilient member 22 may for example be a coil spring. The resilient member 22 may for example be arranged to extend inside the delivery member cover 20 between a radial surface of the delivery member cover 20 and a radial surface provided inside the housing 12.

Turning now to FIGS. 2A to 2C, the cap 14 is shown in more detail. As previously detailed, the cap 14 comprises a shell 14-1 having a distal portion 14-1-1 configured to wrap around the proximal portion 12-1 of the housing 12 for a removable attachment to the housing 12, for instance via a snap-fit or a friction-fit attachment.

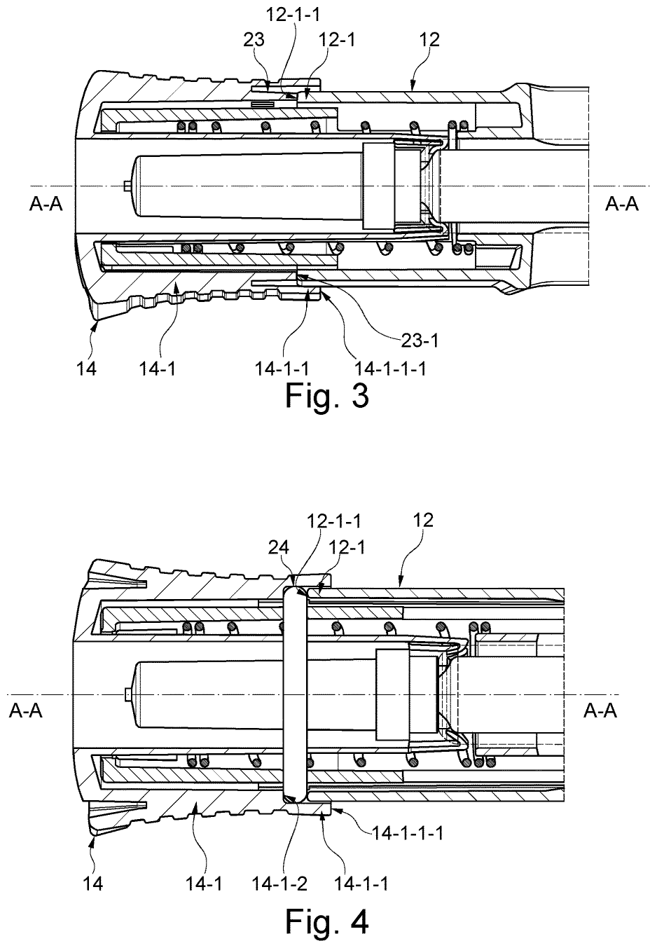

According to a first embodiment of the invention, the shell 14-1 of the cap 14 comprises four flexible ribs 23 extending parallel to the longitudinal axis A-A. The flexible ribs 23 extend parallel to the distal portion 14-1-1 of the shell 14-1 of the cap and radially inward compared to said distal portion 14-1-1, that is to say closer to the longitudinal axis A-A compared to said distal portion 14-1-1. As depicted in FIG. 3, when the cap 14 is attached to the housing 12, a distally facing surface 23-1 of each flexible rib 23 abuts a proximally facing surface 12-1-1 of the proximal portion 12-1 of the housing 12.

The ribs 23 being flexible, they are configured to deform in case of a front end drop of the device, that is to say the side where the cap is attached, or proximal side 10-1. The ribs 23 may be configured to flex radially inward or radially outward. Thereby, the ribs 23 are configured to dampen wave shocks at the interface between the shell 14-1 of the cap 14 and the proximal portion 12-1 of the housing 12. Thus, the ribs 23 prevent force from being transmitted from the cap 14 to the housing 12, or at least only transmit attenuated force to the housing 12.

The ribs 23 form an axial resilient interface area between the cap 14 and the housing 12, axial being relative to the longitudinal axis A-A of the housing. Consequently, the flange 16-2 of the syringe 16, held within the housing 12 by a support 12-3 that is an integral part of the housing 12, does not receive said shock waves, or receives an attenuated version of the shock waves, which prevents the flange 16-2 from breaking.

In the present embodiment, the cap 14 comprises four ribs 23 circumferentially distributed relative to the longitudinal axis A-A, and equally distant from each other, as visible in FIG. 2B. However, in another embodiment, the number of ribs may be lower or higher, and/or the ribs may not be equally distant from each other.

FIG. 4 shows a cross-sectional view of a proximal part of a drug injection device 10 according to a second embodiment of the invention. The second embodiment differs from the first embodiment in that the shell 14-1 of the cap 14 does not comprise any flexible ribs, and instead the drug injection device 10 comprises a resilient ring 24.

Axially, the resilient ring 24 is biased on one side against a distally facing surface 14-1-2 on the shell 14-1 of the cap 14, and on the other side against the proximally facing surface 12-1-1 of the proximal portion 12-1 of the housing 12. The distally facing surface 14-1-2 on the shell 14-1 of the cap 14 is radially closer to the longitudinal axis A-A than the distal portion 14-1-1 of the shell 14-1 of the cap 14. Moreover, the distally facing surface 14-1-2 on the shell 14-1 of the cap 14 is axially closer to the proximal side 10-1 of the drug injection device 10 than a distally facing surface 14-1-1-1 of the distal portion 14-1-1 of the shell 14-1 of the cap 14.

Radially, the resilient ring 24 is biased against the distal portion 14-1-1 of the shell 14-1 of the cap 14, and is closer to the longitudinal axis A-A than said distal portion 14-1-1.

The resilient ring 24 is preferably made of rubber or thermoplastic elastomer but may be made of any other resilient material able to compress in order to dampen force transmittal.

The resilient ring 24 forms an axial resilient interface area between the cap 14 and the housing 12, axial being relative to the longitudinal axis A-A of the housing. Consequently, the flange 16-2 of the syringe 16, held within the housing 12 by a support 12-3 that is an integral part of the housing 12, does not receive shock waves in case of a front-end drop of the drug injection device 10, or at least receives an attenuated version of said shock waves, which prevents the flange 16-2 from breaking.

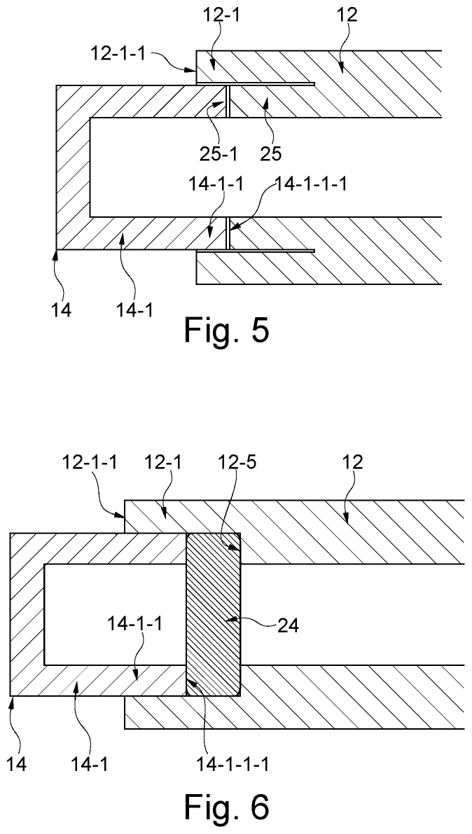

FIG. 5 shows a simplified schematic view of a cap 14 and a housing 12 of a drug injection device 10 according to a third embodiment of the invention.

The third embodiment differs from the first embodiment in that the proximal portion 12-1 of the housing 12 wraps around the distal portion 14-1-1 of the shell 14-1 of the cap 14 for removable attachment of the cap 14 to the housing 12, for instance via snap-fit or friction-fit. The third embodiment also differs from the first embodiment in that some flexible ribs 25 are now part of the housing 12, not part of the cap 14.

The general principle remains the same. The flexible ribs 25 extend parallel to the proximal portion 12-1 of the housing 12 and radially inward compared to said proximal portion 12-1, that is to say closer to the longitudinal axis A-A. When the cap 14 is attached to the housing 12, a proximally facing surface 25-1 of each flexible rib 25 abuts the distally facing surface 14-1-1-1 of the distal portion 14-1-1 of the shell 14-1 of the cap 14.

The ribs 25 being flexible, they are configured to deform in case of a front end drop of the device, that is to say the side where the cap is attached, or proximal side 10-1. The ribs 25 may be configured to flex radially inward or radially outward. Thereby, the ribs 25 are configured to dampen wave shocks at the interface between the shell 14-1 of the cap 14 and the housing 12. Thus, the ribs 25 prevent wave shocks from being transmitted from the cap 14 to the housing 12, or at least only transmit attenuated wave shocks to the housing 12.

The ribs 25 form an axial resilient interface area between the cap 14 and the housing 12, axial being relative to the longitudinal axis A-A of the housing. Consequently, the flange 16-2 of the syringe 16, held within the housing 12 by a support 12-3 that is an integral part of the housing 12, does not receive a shock wave, or receives an attenuated version of said shock waves, which prevents the flange 16-2 from breaking.

FIG. 6 shows a simplified schematic view of a cap 14 and a housing 12 of a drug injection device 10 according to a fourth embodiment of the invention. The fourth embodiment differs from the second embodiment in that the proximal portion 12-1 of the housing 12 wraps the distal portion 14-1-1 of the shell 14-1 of the cap 14 for removable attachment of the cap to the housing, for instance via snap-fit or friction-fit.

Axially, the resilient ring 24 is biased on one side against the distally facing surface 14-1-1-1 of the distal portion 14-1-1 of the shell 14-1 of the cap 14, and on the other side against a proximally facing surface 12-5 of the housing 12. The proximally facing surface 12-5 of the housing 12 is radially closer to the longitudinal axis A-A than the proximal portion 12-1 of the housing 12.

Moreover, the proximally facing surface 12-5 of the housing 12 is axially closer to the distal side 10-2 of the drug injection device 10 than the proximally facing surface 12-1-1 of the proximal portion 12-1 of the housing 12.

Radially, the resilient ring 24 is biased against the proximal portion 12-1 of the housing, radially closer to the longitudinal axis A-A than said proximal portion 12-1.

The general principle remains the same. The resilient ring 24 forms an axial resilient interface area between the cap 14 and the housing 12, axial being relative to the longitudinal axis A-A of the housing. Consequently, the flange 16-2 of the syringe 16, held within the housing 12 by a support 12-3 that is an integral part of the housing 12, does not receive shock waves in case of a front-end drop of the drug injection device 10, or at least receives an attenuated version of said shock waves, which prevents the flange 16-2 from breaking.

Various modifications to the embodiments described are possible and will occur to those skilled in the art without departing from the invention which is defined by the following claims.

The drug injection devices described herein can be used for the treatment and/or prophylaxis of one or more of many different types of disorders. Exemplary disorders include, but are not limited to: rheumatoid arthritis, inflammatory bowel diseases (e.g. Crohn's disease and ulcerative colitis), hypercholesterolaemia, diabetes (e.g. type 2 diabetes), psoriasis, migraines, multiple sclerosis, anaemia, lupus, atopic dermatitis, asthma, nasal polyps, acute hypoglycaemia, obesity, anaphylaxis and allergies. Exemplary types of drugs that could be included in the drug injection devices described herein include, but are not limited to, antibodies, proteins, fusion proteins, peptibodies, polypeptides, pegylated proteins, protein fragments, protein analogues, protein variants, protein precursors, and/or protein derivatives. Exemplary drugs that could be included in the drug delivery devices described herein include, but are not limited to (with non-limiting examples of relevant disorders in brackets): etanercept (rheumatoid arthritis, inflammatory bowel diseases (e.g. Crohn's disease and ulcerative colitis)), evolocumab (hypercholesterolaemia), exenatide (type 2 diabetes), secukinumab (psoriasis), erenumab (migraines), alirocumab (rheumatoid arthritis), methotrexate (amethopterin) (rheumatoid arthritis), tocilizumab (rheumatoid arthritis), interferon beta-1a (multiple sclerosis), sumatriptan (migraines), adalimumab (rheumatoid arthritis), darbepoetin alfa (anaemia), belimumab (lupus), peginterferon beta-1a′ (multiple sclerosis), sarilumab (rheumatoid arthritis), semaglutide (type 2 diabetes, obesity), dupilumab (atopic dermatis, asthma, nasal polyps, allergies), glucagon (acute hypoglycaemia), epinephrine (anaphylaxis), insulin (diabetes), atropine and vedolizumab (inflammatory bowel diseases (e.g. Crohn's disease and ulcerative colitis)). Pharmaceutical formulations including, but not limited to, any drug described herein are also contemplated for use in the drug injection devices described herein, for example pharmaceutical formulations comprising a drug as listed herein (or a pharmaceutically acceptable salt of the drug) and a pharmaceutically acceptable carrier. Pharmaceutical formulations comprising a drug as listed herein (or a pharmaceutically acceptable salt of the drug) may include one or more other active ingredients, or may be the only active ingredient present.

1-9. (canceled)

10. A drug injection device, comprising a cap and a housing, said cap being removably attached to a proximal end of the housing, the housing comprising a support configured to axially abut a flange of a syringe,
wherein the cap comprises at least one flexible rib extending axially, a distally facing surface of said flexible rib abutting a proximally facing surface of the housing.

11. The drug injection device according to claim 10, wherein there is more than one such flexible rib, said flexible ribs being circumferentially distributed around a longitudinal axis of the drug delivery device.

12. The drug injection device according to claim 11, comprising four such flexible ribs equally distant from each other

13. A drug injection device, comprising a cap and a housing, said cap being removably attached to a proximal end of the housing, the housing comprising a support configured to axially abut a flange of a syringe,
wherein the housing comprises at least one flexible rib extending axially, a proximally facing surface of said at least one flexible rib abutting a distally facing surface of the cap.

14. The drug injection device according to claim 13, wherein there is more than one such flexible rib, said flexible ribs being circumferentially distributed around a longitudinal axis of the drug delivery device.

15. The drug injection device according to claim 14, comprising four such flexible ribs equally distant from each other.

16. A drug injection device, comprising a cap and a housing, said cap being removably attached to a proximal end of the housing, the housing comprising a support configured to axially abut a flange of a syringe,
wherein the drug injection device comprises a resilient ring located between a distally facing surface of the cap and a proximally facing surface of the housing.

17. The drug injection device according to claim 16, wherein the resilient ring is made of rubber or a thermoplastic elastomer.

18. The drug injection device according to claim 16, wherein the cap comprises a distal portion shrouding a proximal portion of the housing, and the ring is radially biased against said distal portion of the cap.

19. The drug injection device according to claim 16, wherein the housing comprises a proximal portion shrouding a distal portion of the cap, and the ring is radially biased against said proximal portion of the housing.

20. The drug injection device according to claim 19, comprising a syringe located within the housing and having a flange held by the support of the housing, said flange axially abutting the support.
